(19) United States
(12) Patent Application Publication
(10) Pub. No.: US 2012/0303058 A1
(43) Pub. Date: Nov. 29, 2012

(54) THERMOFORMING PROCESS AND PRODUCTS OBTAINABLE BY THE PROCESS

(75) Inventors: Mikko Huttunen, Tampere (FI); Kimmo Lähteenkorva, Tampere (FI); Anssi Mustonen, Tampere (FI)

(73) Assignee: CONMED LINVATEC BIOMATERIALS OY LTD, Tampere (FI)

(21) Appl. No.: 13/513,455

(22) PCT Filed: Dec. 3, 2010
(86) PCT No.: PCT/FI2010/050997

§ 371 Date: Aug. 14, 2012

Related U.S. Application Data
(60) Provisional application No. 61/266,824, filed on Dec. 4, 2009.

(30) Foreign Application Priority Data
Dec. 4, 2009 (FI) 20096285

(51) Int. Cl. B29C 43/18; A61B 17/04

(52) U.S. Cl. 606/232; 264/271.1

(57) ABSTRACT

The invention concerns composite surgical devices and a method of manufacturing thereof. The device comprises a tissue fixation implant and a protruding member attached to the implant. The method comprises providing a polymeric implant preform comprising a fixation zone for the protruding member, inserting a protruding member into the fixation zone of the preform, inserting the preform into a mold cavity corresponding to desired shape of the tissue fixation implant and comprising at least one orifice for receiving the protruding member and subjecting the preform to heat and pressure for giving the tissue fixation implant the desired shape and attaching the protruding member to the tissue fixation implant.

### FIELD OF THE INVENTION

The invention relates to a novel thermoforming process in particular for the manufacture of surgical composite structures, such as tissue fixation implants. The implants are commonly referred to as anchors because they generally anchor a suture to the target tissue. In addition, the invention relates to novel surgical structures obtainable by the process.

### BACKGROUND OF THE INVENTION

Tissue fixation implants generally function as suture anchors, thus providing an attachment spot for a suture in a desired tissue. The suture can, for example, be joined with a needle extending from its end and the implant is joined to some other point of the suture, for example at the other end of the suture. In addition, implants can be joined with sutures or other distinct members to form other kinds of surgical devices.

Tissue fixation implants of the present kind are conventionally manufactured by injection molding. U.S. Pat. No. 5,964,783 (Grafton et al.) discloses an injection molded suture anchor with insert suture. The suture anchor comprises a biodegradable polymer body molded around a loop of suture, the body shaped so as to have a drive head and screw thread spirals. The suture comprises irregularities, such as knots, which hold the suture within the body. The anchor is manufactured by insert-molding, i.e. by placing the suture within an injection mold and injecting polymer into the mold. U.S. Pat. No. 7,226,469 (Benavitz et al.) discloses another insert-molded suture anchor.

A problem associated with prior art is that the natural adhesion of the suture and an injection molded implant is in many applications not mechanically sufficient for the application and therefore knots or loops are needed within the implant to hold the suture in place. Knots or loops are, however, not always desired or even possible to use. A knot may decrease the tensile strength of the suture and limit the maximum tension force to which the suture may be subjected. On the other hand, the intended use or the implant may not allow use of a suture loop. For example, in some designs (such as shown in FIG. 1 I), the suture must run into the implant from one end thereof and out of the implant from its side.

Another disadvantage of known melt processing techniques is that most known suture materials do not withstand high processing temperatures that may be required by these. For example, the mechanical properties of ultra high molecular weight polyethylene sutures are considerably weakened at temperatures above about 110° C.

### SUMMARY OF THE INVENTION

It is an aim of the invention to solve at least part of the above problems and to provide a novel manufacturing process for surgical composite devices, combining two or more distinct members, such as a polymeric implant body and a suture. It is also an aim to provide corresponding surgical composite devices. A particular aim of the invention is to provide composite surgical devices in which the fixation force between the distinct members in the implant is high.

An aim of the invention is also to provide a manufacturing process which can be used for a wider scope of materials of the distinct members and their combinations, in particular with respect to their heat stability.

In particular, it is an object of the invention to provide a manufacturing method which can be used for manufacturing surgical composite devices having a self-reinforced structure, and a corresponding surgical device.

The invention is based on the idea that the distinct members are affixed to each other in a thermoforming process simultaneously giving one of the members, i.e., the tissue fixation implant, its final shape.

Thus, the method according to the invention for manufacturing a composite surgical device comprising a tissue fixation implant and a protruding member, such as a suture, attached to the implant comprises
providing a polymeric implant preform comprising a fixation zone for the protruding member,
inserting a protruding member into the fixation zone of the preform,
inserting the preform into a mold cavity corresponding to a desired shape of the tissue fixation implant and comprising at least one orifice for the protruding member,
subjecting the preform to heat and pressure for providing the tissue fixation implant the desired shape and for attaching the protruding member to the tissue fixation implant.

According to a typical embodiment, the protruding member is flexible. In particular, the protruding member can be a suture or similar fibrous element. However, other biocompatible members, in particular braided structures can also be joined with the implant using the present method. The protruding member can also be rigid, such as a self-reinforced rod or a similar element.

According to one aspect, the composite surgical device according to the invention comprises
a bioabsorbable tissue fixation implant, and
a protruding member, such as a suture, integrally joined with the bioabsorbable tissue fixation implant,
   wherein the protruding member is joined with the bioabsorbable tissue fixation implant in a thermoforming process under heat and pressure.

According to a second aspect, the composite surgical device comprises a bioabsorbable tissue fixation implant and a suture, in particular a knotless suture, integrally joined with the bioabsorbable tissue fixation implant, wherein the pull-out force of the suture from the implant is equal or close to tensile strength of suture.

According to one embodiment, the suture is joined at one end with a surgical needle to form a surgical kit.

More specifically, the invention is defined in the independent claims.

The invention provides significant advantages. In particular, thermoforming has been found to firmly attach sutures and the like braided structures to implants. That is, the pull-out strength of the suture from the implant is high. This is evidenced by way of examples later in this document. It is also an advantage of the invention that the thermoforming process is by nature solvent-free, extending the scope of materials that can be used for the implant and for the suture.

In particular, many biostabile high-temperature polymers, such as UHMWPE and PEEK, are difficult to affix to an implant by conventional techniques but can be processed using the present thermoforming method because there is no need to reach the melting temperature of the polymer or the deformation temperature of the protruding member (which is usually less than the melting temperature of many bio stabile polymers).

A composite surgical device wherein the protruding member is a suture and the pull-out tensile force of the suture from the implant is 35 N or more, and even 40 N or more, can be manufactured using the method according to the invention.

In addition to merely fixing the suture or other protruding member to implant, a high-quality implant can be produced in the same process. For example, defects on surface of the implant can be avoided in a thermoforming process. As the temperature of the preform material is kept typically below its melting point, the material, due to its high viscosity, is not prone to exit the mold cavity through the mold seams or suture orifice(s). However, it is preferred that the distance between the inner wall of the orifice(s) and the suture is 0.1 mm at maximum.

The method of the invention differs from injection molding and other melt processing methods, such as extrusion, insert injection molding, transfer molding etc. In the present method processing temperatures can be kept relatively low, enabling material combinations which are not possible by using conventional melt processing method. As complete melting is not required or desired in the present method, the present manufacturing process can be carried out at low temperatures, for most biocompatible implant materials at temperatures below 150° C. Even lower temperatures can be used (for example because of the durability of the suture), provided that a preform material having a glass transition temperature low enough is chosen. Thus, materials and material combinations can be used where one or all components are temperature sensitive or where components are not otherwise compatible or processable in molten form. Therefore, high strength of both implant and suture materials can be maintained in the process. To mention only one example, the method is well suited for ultra high molecular weight polyethylene sutures not compatible with melt processing, in which case the thermoforming temperature is preferably less than 110° C.

One advantage of the invention is that biodegradable polymers can be used as implant and/or suture material. Processing at low temperatures maintains the molecular mass of the polymer. In other words, chemical degradation of the polymer in not initiated in the process.

Moreover, the present invention makes it possible to produce self-reinforced implant structures having strong bonding with the suture, provided that the implant preform is made from self-reinforced material. That is, thermoforming does not cause self-reinforcement to relax, provided that the temperature is kept sufficiently low (typically a temperature below Tₘ of the preform is sufficiently low) and provided that the process is fast enough and the preform is under compression during the heating phase of thermoforming. It is also beneficial if the compression distance or duration during the compression phase is relatively short in order not to cause the reinforcing structure of the preform to lose its structure at least completely. Thus, in general, at least 10%, preferably at least 30%, most suitably at least 50% of the self-reinforcement is maintained in the process.

The present thermoforming process can be easily automated and is well suited for mass production.

The term “composite surgical device” refers to any such surgical device that is manufactured from two or more parts, typically made of different materials or at least in different processes. Typically, the implant is made of first polymer or polymer blend and the suture or other member is made of a second polymer or polymer blend.

The terms “(tissue fixation) implant” and “implant body” refer to any biocompatible and thermoformable body that can be left temporarily or permanently within human or animal tissue. In typical applications, the implant is relatively small, preferably having a maximum dimension of 2 cm or less. In particular, the implant may be an implant used in reconstructive surgery for holding desired tissue in a desired shape or in place with respect to other tissues. Large application areas of the invention are in the fields of sports medicine and trauma surgery, in particular arthroscopic surgery. For example, the present surgical device may be or form part of a meniscal repair device, suture anchor of any kind, or cross-pin ACL fixation device.

The term “protruding member” refers to any member attachable to the implant by the present process such that it extends from the outer surface of the implant to serve a particular surgical purpose, in particular attachment of the implant to another implant, surgical needle and/or tissue. Most importantly, the term includes various surgical sutures, fibers and fibrous elements. However, also rigid protruding members in their final form or as a preform can be used. Not only the implant body but also the protruding member can be shaped by thermoforming in the process. The protruding member can have a self-reinforced or unreinforced structure.

The term “fixation zone” means a region, such as a cannulation in or groove on the polymer preform serving to receive the suture before the thermoforming stage is begun. During the thermoforming stage, the fixation zone deforms and intimately mates with and bonds to the suture such that the desired fixation of the suture and the implant is achieved.

The term “defomation temperature” of a protruding member means a temperature where the protruding member, such as a suture, starts to permanently lose its strength, in particular tensile strength, due to irreversible chemical or physical processes. Usually, this temperature corresponds to the glass transition temperature T_{g} of the suture material.

Further embodiments and advantages of the invention will now be described in detail with reference to the attached drawings.

### BRIEF DESCRIPTION OF THE DRAWINGS

FIGS. 1A-1I show in perspective views different types is composite devices, including a suture or a plurality of sutures and an implant that can be manufactured according to the invention.

FIG. 2 shows in an exploded perspective view a mold cavity, an implant preform with suture and a thermoformed composite device according to one embodiment of the invention.

FIG. 3 shows in a perspective view molding arrangement according to one embodiment of the invention.

FIG. 4 shows in a detailed cross-sectional perspective view of mold means according to one embodiment of the invention.

FIGS. 5A and 5B show a schematic view of the behavior of the self-reinforcing internal structure of the implant using conventional machining and the present method, respectively.

FIG. 6 shows a first set of results of thermoforming experiments for connecting a non-absorbable suture and bioabsorbable polylactide implant.

FIG. 7 shows a second set of results of thermoforming experiments for connecting a non-absorbable suture and bioabsorbable polylactide implant.

FIG. 8 illustrates a third set of pull out force experiment results made with a polylactide implant and HiFi suture (UHMWPE).

FIG. 9 shows the results of an endurance test made with polylactide implants containing polyester and HiFi sutures (UHMWPE).

### DETAILED DESCRIPTION OF EMBODIMENTS

The invention describes a solvent-free method to connect two distinct members into single composite structure by thermoforming, i.e., using heat and pressure. In the following description, a suture and bioabsorbable tissue fixation implant preform, are frequently used as examples of the distinct members. The preform forms the implant after thermoforming. However, it must be noted that the invention allows also other types of member to be joined, as mentioned above and will be described also later in this document.

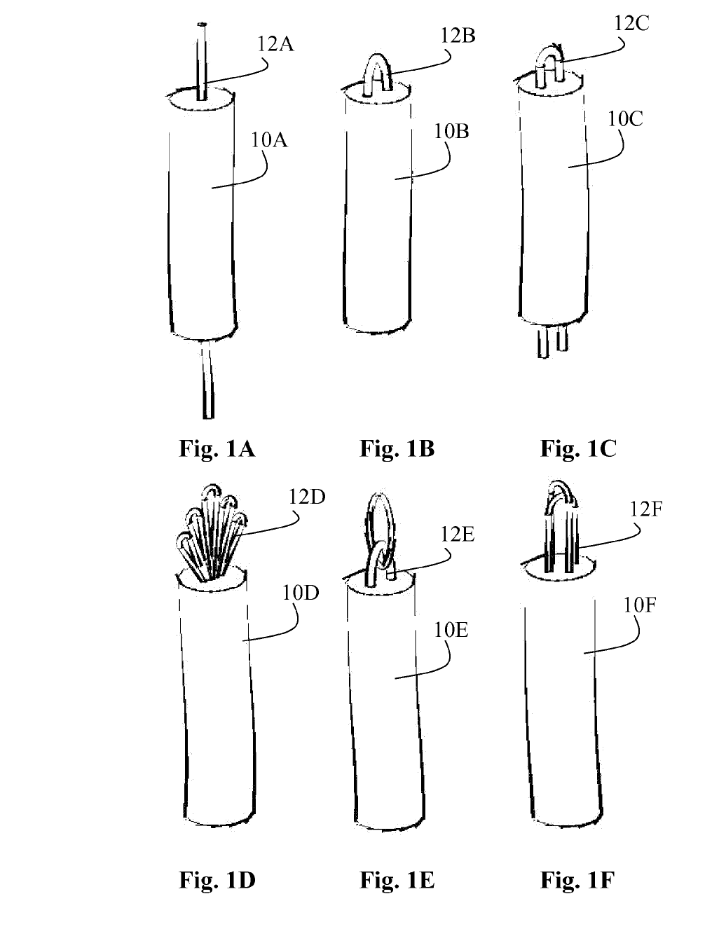

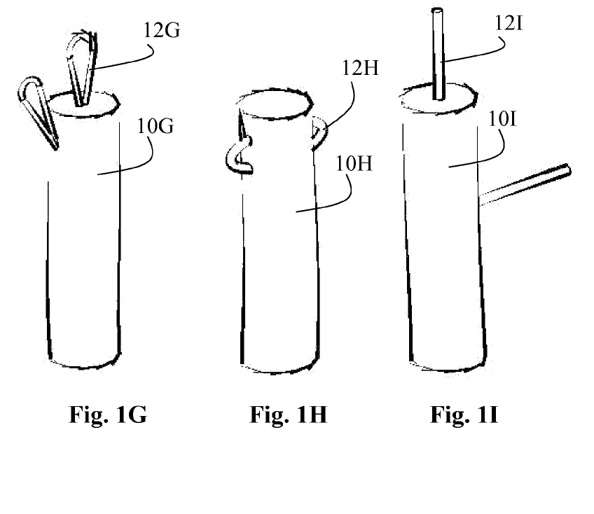

The composite product according to the invention may be a suture anchor or some other braid/bioabsorbable body arrangement connecting several implant parts together. Typically, the implant or at least one of several implants of the suture arrangement is an anchor-type part designed to be immobilized to a tissue, such as bone, cartilage, skin, muscle or internal organ, for allowing binding of the tissue to another tissue location using for example a suture or metal implant. A non-comprehensive list of examples of products where the invention can be utilized includes various forms of suture anchors, implants containing a continuous suture, implants containing an endless braid or fiber loop, implants having a self-reinfoced and/or oriented body, and implants having a textured body surface. FIGS. 1A-1I show several examples of embodiments of the invention. FIG. 1A shows a simple structure comprising a single suture 12A running through a thermoformed implant 10A. This kind of device is preferably manufactured using a implant preform comprising a through-hole into which the suture 12A is inserted. The thermoforming mold associated with this embodiment comprises respectively two orifices for respective protruding portions of the suture 12A (see also detailed description of the process below). FIG. 1B shows an implant 10B having a suture loop 12B provided on one end thereof. The loop may be endless or there may suture ends “free” within the implant 10B. The loop may be used to bind a separate suture or harness (not shown) to the implant. FIG. 1C shows an implant where the suture branches of the suture loop 12C are guided through the implant 10C. FIG. 1D shows an implant 10D having several suture loops 12D formed in a fan-like manner on one end thereof. The loops may be formed of a single suture or a plurality of sutures. FIG. 1E shows a device according to FIG. 1B with the difference that there is threaded a separate suture loop to the suture loop 12E thermoformed to the implant 10E. FIG. 1F shows an implant 10F comprising two cross-installed suture loops 12F on one end thereof. FIG. 1G shows an implant having in addition to an end-installed suture loop 12G a side-installed suture loop. FIG. 1H shows an implant 10H comprising two side-installed suture loops 12H, which, however, may be formed of only one endless suture loop partially buried to the implant 10H. FIG. 1I shows an implant 10I having a through-passing suture 12I. The suture 12I enters the implant on end thereof and exits on side wall thereof at essentially right angle.

FIG. 1A-1I show only some exemplary embodiments that may be implemented using the present process. As appreciated by a person skilled in the art, several variations and combinations of the abovementioned examples are possible.

FIGS. 1A-1I show a simple cylindrical implant without any surface texture or additional shaping. In many practical applications, the implant is textured. There may be provided on the implant grooves, protrusions, pits, bumps, screw thread, or the like structures, depending on the intended use of the device. According to one embodiment, the implant comprises a directed texture allowing the movement of the implant in tissue better in one direction than the other.

Generally speaking, both the implant and the suture can be either biostabile or bioabsorbable. However, typically at least the implant is bioabsorbable for removing the need of a separate removal operation.

At least the following implant types can be manufactured using the invention:
plain suture thermoformed with an absorbable implant body,
knotted plain suture thermoformed with an absorbable implant body,
plain suture with internally attached or penetrating additional locking elements (or other additional components) thermoformed with absorbable implant body,
co-braided (multi components) suture thermoformed with an absorbable implant body,
modified suture (such as impregnated with polymer solution or polymer melt) thermoformed with an absorbable implant body,
modified suture (such as braiding modifications, bifurcations, openings, additional components or loops) thermoformed with an absorbable implant body
combination of two or more of the above-listed examples.

The suture is preferably braided, i.e. a multifilament suture. This increases the strength of the suture. In addition, the adhesion between the implant thermoformed according to the present process is increased as compared with plain sutures, as the polymer fills inter-filament microstructures on the surface of the suture. The suture material can be natural or artificial, for example, polyethylene, polypropylene, polyester, polyetheretherketone (PEEK) or Ultra High Molecular Weight polyethylene (UHMWPE), nylon, silk, steel or blend thereof (non-absorbable suture) or polyglycolic acid, polylactic acid, caprolactone or blend thereof or catgut (absorbable suture). According to a preferred embodiment, the suture is polymeric. The suture may be coated or uncoated. In principle, all commercially available surgically usable suture thread types, whether biodegradable or biostabile, can be used within the invention.

The implant, i.e., preform material comprises or essentially consists of thermoplastic polymer or polymer blend. A non-comprehensive list of bioabsorbable (resorbable) polymers, copolymers and terpolymers which may be utilized to manufacture bioabsorbable polymeric fibers and bioabsorbable polymeric bodies usable within the invention comprises: polyglycolide (PGA), copolymers of glycolide: glycolide/L-lactide copolymers(PGA/PLLA) glycolide/trimethylene carbonate copolymers (PGA/TMC); polylactides (PLA) stereocopolymers of PLA: poly-L-lactide (PLLA) poly-DL-lactide (PDLLA) L-lactide/DL-lactide copolymers, other copolymers of PLA: lactide/tetramethylglycolide copolymers, Lactide/trimethylene carbonate copolymers, lactide/d-valerolactone copolymers, lactide/[epsilon]-caprolactone copolymers, terpolymers of PLA: lactide/glycolide/trimethylene carbonate terpolymers, lactide/glycolide/[epsilon]-caprolactone terpolymers, PLA/polyethylene oxide copolymers, polydepsipeptides, unsymmetrically 3,6-substituted poly-1,4-dioxane-2,5-diones, polyhydroxyalkanoates: polyhydroxybutyrates (PHB), PHB/b-hydroxyvalerate copolymers, (PHB/PHV) poly-b-hydroxypropionate (PHPA), poly-p-dioxanone (PDS), poly-d-valerolactone-poly-e-caprolactone, methylmethacrylate-N-vinyl pyrrolidone copolymers, polyesteramides, polyesters of oxalic acid polydihydropyrans-polyalkyl-2-cyanoacrylates, polyurethanes (PU), polyvinylalcohol (PVA), polypeptides, poly-b-malic acid (PM LA), poly-b-alkanoic acids, polycarbonates, polyorthoesters, polyphosphates, polyanhydrides, and tyrosine derived polycarbonates, polyetherketone (PEK), polyetheretherketone (PEEK), polyetherketoneketone (PEKK), polyetheretherketoneketone (PEEKK), polyetherketoneetherketone (PEKEK) or Ultra High Molecular Weight polyethylene (UHMWPE), -propylene (UHMWPP), or derivatives, copolymers or mixtures thereof. In addition, composites of a bioactive component and polymer can be used. The bioactive component can comprise, for example, bioactive bioceramic and/or glass, hydroxyapatite (HA), other calcium phosphates, such as tricalcium phosphates (TCP), combinations of different calcium phosphates, such as HA/TCP, calcium carbonate and/or calcium sulphate.

The adhesion of the suture to the implant may be even increased by using an adhesion-promoting agent which is applied to the contact zone of the suture and the preform or mixed with the preform material. For example, polymer with lower melting point than the preform material (or totally amorphous polymer) and/or a polymer with lower molar mass than the preform material can be used as an adhesion promoter between the polymeric implant body and the suture.

According to one embodiment, the implant is manufactured from self-reinforced polymer preform material, i.e. material containing a specific molecular orientation and/or reinforcing component increasing its strength at least in one torsional or tensile direction. Torsional strength in a longitudinal direction is of particular importance in structures similar to that presented in FIG. 1I and similar implants subjected to transverse forces during use. If the surface of the implant is textured during thermoforming, the self-reinforcing structure is maintained in the textures provided that the relaxation of the preform is limited or controlled. For example, it is preferable that the compression length is sufficiently short and the temperature less than the melting point of the self-reinforced polymer. Preferably, a self-reinforced preform is compressed less than 20%, in particular less than 10% of the initial length of the preform in the direction of compression during said subjecting the preform to heat and pressure. In addition, the preform material is preferably kept under tensional, compressional or the like force during the thermoforming cycle.

The compression duration is preferably less than 10 min, in particular less than 60 s, typically 1-60 s.

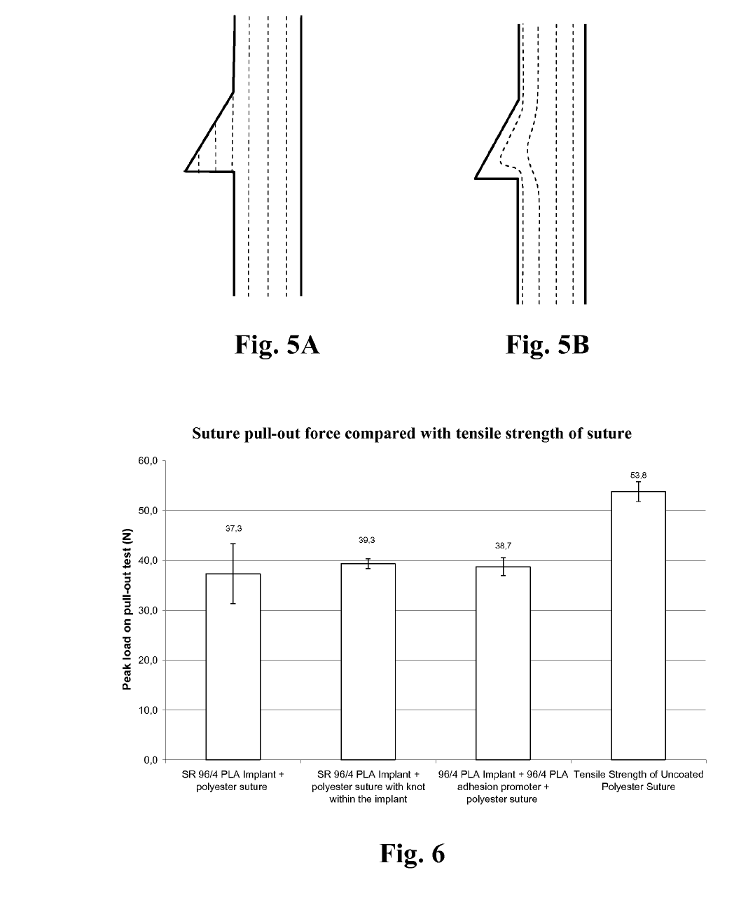

FIGS. 5A and 5B illustrate the difference between conventional self-reinforced implants and self-reinforced implants made taking advantage of the invention. In the conventionally machined implant of FIG. 5A, the self-reinforcement illustrated using the dash lines is interrupted at the region of the protrusion of the implant. A thermoformed implant shown in FIG. 5B, maintains the continuity of the self-reinforcement and thus produces a stronger protrusion. The same applies for other shapes, such as grooves.

Self-reinforcing of the implant preform can be achieved e.g. by solid state deformation, like with free or die drawing, biaxial drawing, compression, hydrostatic extrusion or ram extrusion as combined with drawing. Orientation and/or self-reinforcing techniques, which can be applied to manufacture the materials of the invention have been described in many publications, like in U.S. Pat. No. 4,968,317, EPO Pat. No. 0423155, EPO Pat. No. 0442911, FI Pat. No. 88111, FI Pat. No. 98136, U.S. Pat. No. 6,221,075 and U.S. Pat. No. 4,898,186, the entire disclosures of which are incorporated herein by way of this reference.

According to one embodiment the thermoformed implant serves to mechanically join two or more separate sutures. In other words, there are at least two sutures brought into the mold cavity before the thermoforming process starts such that they are all affixed to the implant by heat and pressure. This kind of implant having several sutures connected into one implant body can be used to connect sutures of the same type or, in particular, of different types. That is, the invention can be used to connect sutures having differing properties with respect to their material, strength, diameter, bioabsorbancy or market price, for example.

According to one embodiment, the present device comprises a bioabsorbable portion (e.g. implant and/or suture portion which is left within the body) and a biostabile portion (e.g. suture portion assisting in insertion/fixation of the device).

The suture can be permanently or impermanently connected, at one or both ends, to a surgical needle in order to form a ready-to-use surgical instrument.

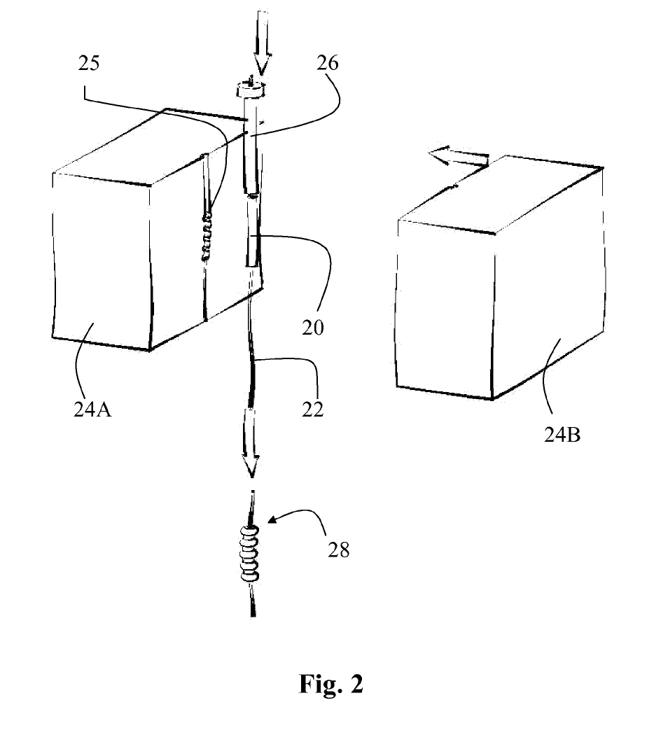

In the following, the thermoforming process and a thermoforming apparatus suitable for carrying out the invention are described in more detail with reference to FIGS. 2-4.

According to one embodiment, the thermoforming process comprises
providing a suture 22,
providing a preform 20 with a cannulation or a fixation zone of other geometrical shape for receiving the suture,
threading the suture 22 to the cannulation or other fixation zone,
placing the preform 20 including the suture 22 into a mold, and
heating the preform 20 under a predetermined pressure and temperature high enough to compress the preform onto suture but not high enough to damage the suture.

According to one embodiment, the mold comprises a heatable first mold portion, and a second mold portion, i.e., a piston, moveable with respect to the first mold portion. For being able to keep the suture within the preform during molding, there is provided at least one orifice residing on the first or second mold portion or, in the case of a through-running suture, both. The heating of the preform is achieved by heating the first mold portion. The pressure, for its part, is achieved by moving the piston with respect to the first mold portion. The piston is typically moved along the direction of the suture for achieving the desired pressure on the mold cavity.

The first mold portion preferably comprises at least two mold halves compressible against each other in a direction perpendicular to the direction of movement of the piston. The mold cavity 25 is defined by mold members, which can be opened. Preferably, the mold comprises two first mold members 24A and 24B, typically providing horizontal compression, and a second mold member 26, providing vertical compression. The suture 22 passes out of the mold cavity through one or more orifices provided at or between the mold members. In a typical embodiment, the second mold member 26, acting as a piston, comprises a through-hole having a diameter slightly (e.g. 0,01-0,2 mm) larger than the diameter of the suture 22.

It is to be noted that the mold arrangement can also be in any other orientation and that the compression directions may vary from oblique to perpendicular. According to another example, the first mold members 24A and 24B are arranged to provide vertical compression and the second mold member 26 horizontal compression. In a still another example, both compressions take place in the horizontal plane.

The compression may also be multidirectional by nature, which is achievable by hydrostatic compression means, for example.

The thermoforming process gives the final design to the whole implant 28 and integrally binds the suture to the implant. Therefore, the mold cavity 25 typically comprises a textured inner wall so as to manufacture a tissue fixation implant 28 having a corresponding surface texture.

According to one embodiment, the method comprises, as the first step, manufacturing the preform itself. This can be carried out by conventional plastic processing methods such as insert or injection molding, extrusion, machining, etc. Preform manufacturing could also include self-reinforcing, which can lead to stronger end-products, because the thermoforming process can be designed so that the majority, or at least significant portion of the self-reinforcing molecular orientation will be maintained during the process.

In the thermoforming process the preform material will generally be heated to a temperature range over T_{g} but below Tₘ, and the final form will be achieved by simultaneous presence of heat and pressure, thus resembling compression molding. For example, for a polylactide implant, the optimal temperature is in the range 65-170° C., in particular 110-150° C. In particular when manufacturing self-reinforced implants, the preform is heated under a predetermined pressure and temperature high enough to compress the preform onto suture but low enough not to relax self-reinforcement of the preform completely.

According to a practical example, the method comprises the following steps:
1. A suture is passed through a central hole of a tubular polylactide billet (preform).
2. The suture is passed through a fine hole of a plunger (piston).
3. The billet is placed inside a mold while keeping the suture tight. If necessary, the other end of the suture is passed through a second hole in the mold cavity.
4. Closing the mold
5. Raising the temperature of the mold to a desired level
6. Applying compressive force to the billet using movement of the plunger (and naturally holding the mold tightly closed).
7. After a desired compression time, the mold is cooled back to room temperature and the mold is opened.

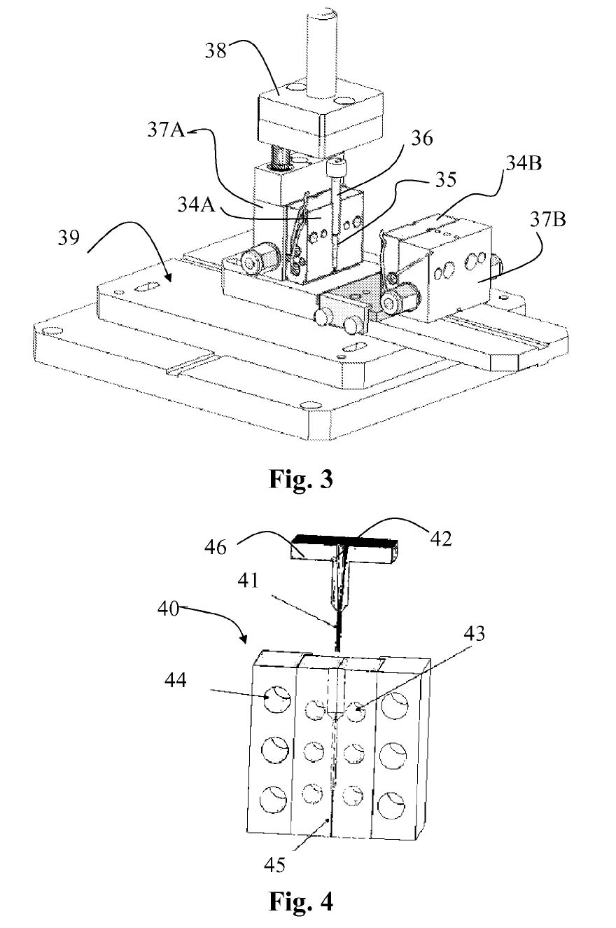

FIG. 3 shows a molding apparatus according to one embodiment of the invention. The apparatus comprises a body 39 having a first holder 37A for a stabile mold member 34A and a horizontally moveable holder 37B for a moveable mold member 34B. The apparatus comprises means for firmly compressing the mold members 34A and 34B against each other. The mold members 34A and 34B, together forming a mold, contain a recess which is shaped so as to form the mold cavity 35 corresponding to the shape of the desired product and to allow a piston 36 to compress the mold cavity in vertical direction. The mold members 34A and 34B contain also bores that are used to heat and cool the mold. For achieving the compression or the piston 36, the apparatus comprises a vertically moveable press 38 positioned above the mold cavity.

FIG. 4 shows the piston 46 and the mold 40 in cross section. The piston 46 comprises a broad flange on upper end thereof and a plunger portion extending from the flange and serving as a plunger for the mold 40. The lower portion comprises a capillary bore 45, preferably of diameter 0,1-1,0 mm, depending on the suture used, for a suture (not shown). The bore is broadened towards the upper end of the piston 46 for allowing easy insertion of the suture through the piston. The upper surface of the piston may comprise a groove (not shown) through which the suture exits when the press (see FIG. 3) is contacted with the piston 46.

The mold 40 comprises a pathway for the piston 46, the actual mold cavity and a capillary bore for the suture. In addition, the mold comprises two sets of bores. The first set 43 is placed in the vicinity of the mold cavity and is designed to receive heating means, such as heating resistors or heated fluid circulation. The second set 44 is placed farther from the mold cavity and are designed to receive cooling means such as cool fluid circulation.

It is to be noted that the suture need not be conveyed to the mold cavity through the piston but may also go through a channel in the main mold halves or between any of these parts. In addition, for manufacturing structures as shown in FIGS. 1B, 1E, 1F, 1G and 1H, it is not necessary to have an “open” channel for the suture to the outside of the mold at all. In these cases it is enough to have internal “closed” channels on the interior wall of the mold, into which the appropriately cut sutures can be inserted before closing the mold so that a portion of the suture remains in touch with or inside the preform.

The following examples are intended to further clarify advantages of the invention.

### Example 1

Several methods to connect non-absorbable suture and bioabsorbable polylactide have been tested and according to these trials the thermoforming process yielded into most favourable results. The first thermoforming trials were made by placing the polylactide billet horizontally between the mold plates. These first trials resulted in lower pull-out force than the tensile strength of the suture (tensile strength was 53.8 N), but the load was on acceptable level, that is, regularly over 35 N (cf. suture tensile strength with knot was less than 30 N). Results of the trials are shown in FIG. 6. As a comparison other tested techniques (including solvent gluing, solutions using wires passing through the suture, etc.) yielded into pull-out forces ranging from 7 up to 35N.

### Example 2

To improve the adhesion between suture (polyester) and polylactide a vertical mold was manufactured to increase molding pressure and to make the process more accurate. The billet was aligned vertically and suture was passed through the plunger (piston), as described in detail above.

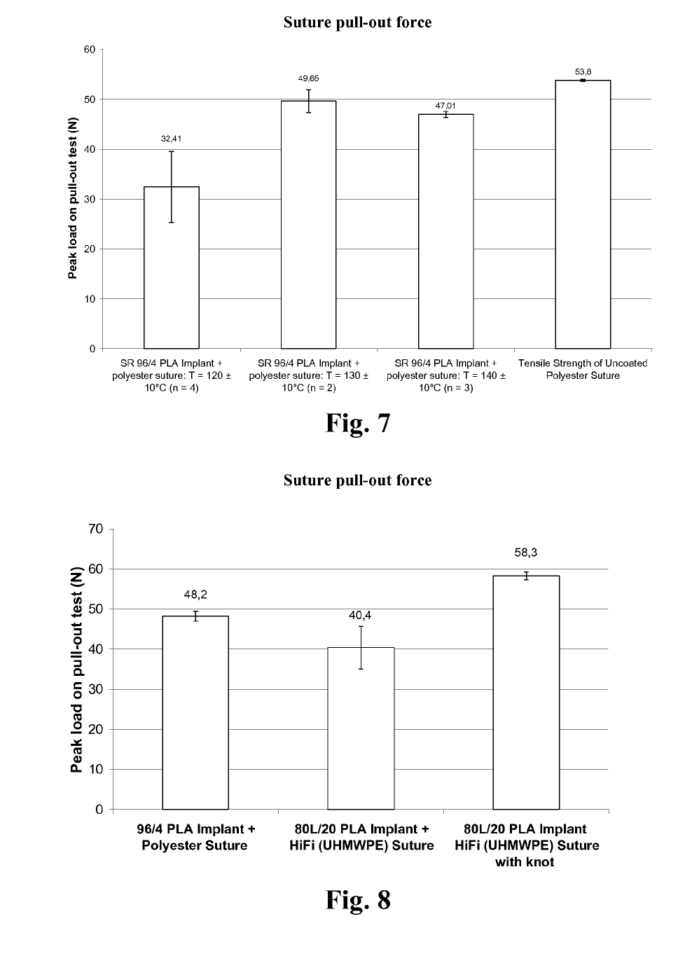

Results of the trials are shown in FIG. 7. By these trials it was proved that it is possible improve the adhesion between polyester suture and polylactide. The maximum pull-out forces for single specimens reached over 50 N (average 49.65 N). Therefore it was shown that adhesion force between implant and suture was comparable/similar to tensile strength of the plain suture. It was also proved that the suture was not damaged during the manufacturing process. At maximum force, the suture breakage was observed without slippage from the inside of the implant body, which indicates good adhesion.

### Example 3

A thermoforming process was done similarly to that presented in Example 2 using polylactide billet and HiFi suture (UHMWPE). Samples of implants having a suture with and without a knot inside the implant body were tested. Results of the trials are shown in FIG. 8. The maximum pull-out forces for single specimens reached near 60 N (Averare 58.3±1 N) for specimens containing a knot inside the implant. Knotless implants with UHMWPE suture demonstrated up to 45 N suture pull-out force for single specimens (average 40.4±5.4N).

The specimens having a knot in the suture within the distal end of the implant demonstrated higher pull-out forces than specimens shown in Example 2.

### Example 4

A thermoforming process was done similarly to that presented in Examples 2 and 3. Polylactide specimens containing both polyester and HiFi suture (UHMWPE) were manufactured and long term adhesion between implant body and suture was analyzed several times within 12 weeks using an in vitro soak study in phosphate buffer solution at 37° C. Polyester suture samples were all knotless, while all HiFi suture specimens included a single knot within the distal end of the implant.

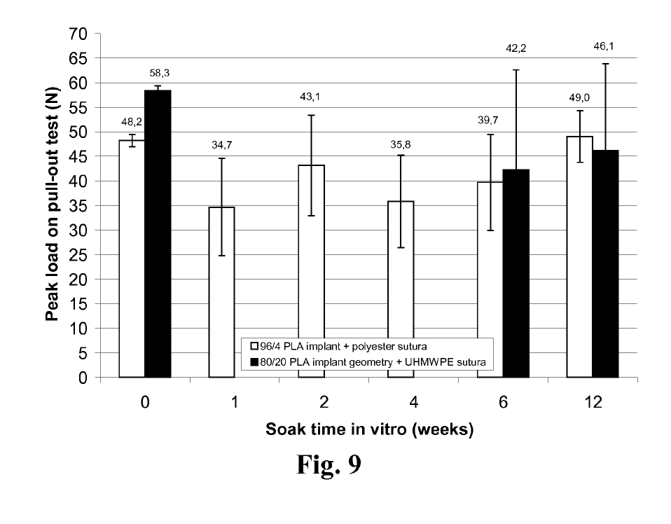

Results of the in vitro study are shown in FIG. 9. According to results both suture/implant material combinations maintained their strength at their initial level throughout the 12 week follow-up. The relatively large standard deviation for measurements shown in the drawings originates from the manual manufacturing process used for making the samples tested.

1. A method of manufacturing composite surgical devices comprising a tissue fixation implant and a protruding member attached to the implant, the method comprising
providing an polymeric implant preform comprising a fixation zone for the protruding member,
inserting a protruding member into the fixation zone of the preform,
inserting the preform into a mold cavity corresponding to a desired shape of the tissue fixation implant and comprising at least one orifice for receiving the protruding member,
subjecting the preform to heat and pressure for providing the tissue fixation implant the desired shape and for attaching the protruding member to the tissue fixation implant.

2. The method according to claim 1, wherein the mold cavity is formed by
a heatable first mold portion, and
a second mold portion moveable with respect to the first mold portion,
said at least one orifice for the protruding member residing on the first or second mold portion or both, and said heating being carried out by heating the first mold portion and said pressure being achieved by moving the second mold portion with respect to the first mold portion.

3. The method according to claim 2, wherein the second mold portion is moved along the direction of protrusion of the protruding member for achieving the pressure.

4. The method according to claim 2, wherein the first mold portion comprises at least two mold halves compressible against each other in a direction oblique or perpendicular to the direction of movement of the second mold portion.

5. The method according to claim 1, wherein the protruding member is a suture or other braided biocompatible member.

6. The method according to claim 1, wherein there is a gap around said protruding member and between said protruding member and said at least one orifice, the minimum gap distance being less than 0.1 mm.

7. The method according to claim 1, wherein the temperature of the implant preform is kept between the glass transition temperature T_{g} and the melting temperature Tₘ of the preform.

8. The method according to claim 1, wherein the implant preform is subjected to a temperature between 65-170° C., typically 65-150° C., in particular 110-150° C.

9. The method according to claim 1, wherein a self-reinforced implant preform is used and the temperature of the preform is kept low enough so as to maintain at least 10% of the self-reinforcement.

10. The method according to claim 9, wherein the preform is compressed during said subjecting the preform to heat and pressure, the compression duration and length being low enough to maintain at least 10%, in particular at least 30% of the self-reinforcement.

11. The method according to claim 1, wherein the implant preform is compressed and kept under pressure for less than 10 min, typically 1-60 s, while heating it.

12. The method according to claim 1, wherein the mold cavity comprises textured inner wall so as to manufacture a tissue fixation implant having a corresponding surface texture.

13. The method according to claim 1, wherein the implant comprises polylactic acid (PLA).

14. The method according to claim 13, wherein the implant comprises self-reinforced polylactic acid (SR-PLA).

15. The method according to claim 1 claim 1, wherein the implant and/or protruding member are/is made from bioabsorbable material.

16. The method according to claim 1, wherein the preform is cylindrical in shape, preferably comprising a central cannulation, groove or other prefabricated formation for receiving the protruding member.

17. The method according to claim 1, wherein the preform is subjected to a temperature which is less than the melting temperature Tₘ of the preform and less than the deformation temperature of the protruding member.

18. The method according to claim 1, wherein the preform is made of biostabile polymer having a melting temperature Tₘ higher than the deformation temperature of the protruding member.

19. The method according to claim 1, wherein the preform is made of material selected from the group of: polyglycolide (PGA), polylactide (PLA), poly-L-lactide (PLLA), polyetheretherketone (PEEK) or Ultra High Molecular Weight polyethylene (UHMWPE), or derivatives, copolymers or mixtures thereof.

20. A composite surgical device, comprising
a tissue fixation implant made of thermoplastic material and
a protruding member integrally formed with the tissue fixation implant,
wherein the protruding member is affixed to the tissue fixation implant in a thermoforming process under pressure and temperature higher than the glass transition temperature T_{g} and lower than the melting temperature Tₘ of the thermoplastic material.

21. The surgical device according to claim 20, wherein the tissue fixation implant is bioabsorbable.

22. The surgical device according to claim 20, wherein the tissue fixation implant is manufactured from self-reinforced material.

23. The surgical device according to claim 20, wherein the protruding member is a surgical suture.

24. The surgical device according to claim 20, being a suture anchor.

25. The surgical device according to claim 20, wherein the preform is made of biostabile polymer having a melting temperature Tₘ higher than the deformation temperature of the protruding member.
